(19) United States
(12) Patent Application Publication
(10) Pub. No.: US 2026/0273258 A1
(43) Pub. Date: Sep. 17, 2026

(54) HAND-HELD POWER TOOL HAVING GRASP-ACTIVATED POWER SWITCH

(71) Applicant: Mark Vincent GRASSANO, Downington, PA (US)

(72) Inventor: Mark Vincent GRASSANO, Downington, PA (US)

(21) Appl. No.: 19/678,043

(22) Filed: May 14, 2026

Related U.S. Application Data
(63) Continuation-in-part of application No. 19/064,669, filed on Feb. 26, 2025.
Application No. 19/064,669 is a continuation-in-part of application No. 17/235,847, filed on Apr. 20, 2021, now abandoned.
(60) Provisional application No. 63/012,772, filed on Apr. 20, 2020.
(60) Provisional application No. 63/922,032, filed on Nov. 20, 2025.

(51) Int. Cl. A61M 37/00

(52) U.S. Cl. CPC A61M 37/0076; A61M 2205/587; A61M 2205/8206

(57) ABSTRACT

A handheld power tool has grip removably attachable to a tool body that encloses a motor. The grip has a grasp-activated power switch. The power tool may have a removable battery attachable to the tool body. A LED band circumvolves either the tool body or the battery. The LED band is powered on and off in response to the grasp-activated power switch and provides a visual indicator of the status of the motor. When the LED band circumvolves the tool body, it is positioned opposite the grip so as to be continuously visible while using the power tool.

### RELATED APPLICATIONS

This application claims priority to U.S. Provisional Application 63/922,032 filed Nov. 20, 2025, and claims priority as a continuation-in-part of U.S. patent application Ser. No. 19/064,669 filed Feb. 26, 2025, which claims priority to and is a continuation-in-part of U.S. patent application Ser. No. 17/235,847 filed Apr. 20, 2021, which claims priority to U.S. Provisional Application 63/012,772 filed on Apr. 20, 2020, all of which are fully incorporated herein by reference.

### BACKGROUND OF THE INVENTION

### Field of the Invention

The present invention relates generally to power switches for hand-held power tools. The invention relates more specifically to a grasp-activated power switch integral to the grip of a hand-held power tool, and most specifically to such a power switch installed on a tattoo machine.

### Description of Related Art

The first tattoo machine was derived from the design for an electric pen invented by Thomas Alva Edison in 1877, for which he was awarded U.S. Pat. No. 196,747. That machine used electromagnetic coils for driving a reciprocating pen and needle through paper to create a perforated pattern for autographic printing. In 1891, pioneering tattoo artist Samuel O'Reilly adapted Edison's device for tattooing by adding a tubular handle for delivering ink from an ink reservoir to the perforating needle. For this design O'Reilly was awarded U.S. Pat. No. 464,801.

From then til now, the basic components of a tattoo machine remain essentially unchanged. Tattoo machines all include some type of chassis adapted for hand-held operation that supports a motive force for actuating a linear motor. The motive force may be electromagnetic coils, a rotary electric motor, or a pneumatic motor. A pen coupled to the motive force draws a supply of tattooing ink to inject the ink into the dermis layer of human skin upon each stroke of the motor. Most innovation for tattoo machines has addressed cosmetic desires or ergonomic concerns to give machines a different look and feel.

One such ergonomic advancement has been the provision of a foot pedal for actuating, i.e. switching on or off, the power supply to the motor of a pen-style tattoo machine. While the foot pedal frees the artist's hands from having to actuate the power switch, power cable running from the foot pedal to the tattoo machine motor can often interfere with the artist's freedom of movement. In addition, having to maintain one foot on or near the foot pedal may cause the tattoo artist to assume an awkward position while tattooing, and in general, frequent actuation of the foot pedal is found by many artists to cause discomfort over time.

To overcome that problem, a more recent ergonomic advantage has been the addition of rechargeable battery packs to the chassis of the tattoo machine, made possible by recent improvements in battery technology. These battery packs are typically mounted to the end of the tattoo machine opposite the needle, which advantageously eliminates interference from a tethered power cord. While such battery packs provide the artist with more freedom of movement in one sense, they introduce new problems that arise from placement of the power switch on the battery pack. Because tattooing is both an operation and an art form that requires surgical precision for finely detailed work, the artist must use both hands in cooperation, whereby one hand (the placement hand) carefully stretches and adjusts the skin of a customer being tattooed to achieve an optimal grip about the tattooing site while the other hand (the operating hand) holds and operates the tattoo machine. During a tattooing session, the artist must frequently start and stop power to the tattoo machine for various reasons including cleaning the skin and conserving battery power during short breaks in the action. But to stop or start power, the artist must remove his placement hand from the customer's skin to push the power switch on the battery pack, and then reposition his placement hand to re-achieve the optimal grip. Such action is inefficient, time consuming, and frustrating to the artist.

For the customer, the frequent movement of the artist's placement hand from the surface of her skin back and forth to the power supply switch can be very disturbing, and is in fact an unsound medical procedure because it creates a potentially unsanitary condition. The artist's placement hand, being in close contact with the customer's perforated and bleeding skin, should remain isolated from all other surfaces that can be a potential source of infection.

What is needed is an efficient way for a tattoo artist to start and stop a hand-held battery-powered tattoo machine without having to assume an uncomfortable posture and without having to release his placement hand from the customer's skin.

### SUMMARY OF THE INVENTION

The present invention discloses an engineered design for a hand-held power tool having a grasp-activated power switch integrated with the grip portion of the tool. Generally, a grasp-activated power switch according to the present invention allows an operator of the tool to switch power on and off to the tool while the tool is being held in the operator's operating hand, by squeezing the gripping portion of the tool handle with the same operating hand. When installed for example on a tattoo machine, a grasp-activated power switch according to the invention allows a tattoo artist to maintain engagement of his placement hand at the tattooing site on a customer's skin while switching power on and off with his operating hand, without releasing his operating hand from holding the machine. The principles of the present invention may have utility in applications other than tattoo machines, for example, in hand-held power tools such as medical instruments, dental instruments, high-speed drills, etc.

In a basic embodiment of the invention, a grasp-activated power switch for a hand-held power tool includes a rigid but slightly flexible outer shell having an inner conductive layer, and an inner channel having an outer conductive layer and located within the outer shell so that the outer conductive layer is electrically separated from the inner conductive layer. The inner channel is configured for attachment to the power tool at a convenient gripping location. A wireless transmitter is electrically coupled to one of the inner channel or the outer shell, and a battery is electrically coupled to another of the inner channel or the outer shell. The power switch is configured so that a manual grasping force applied to the outer shell causes the outer shell to flex inward, and the inner conductive layer to electrically couple to the outer conductive layer to thereby energize the wireless transmitter by electrically coupling the wireless transmitter to the battery.

In a more detailed embodiment, a grasp-activated power switch of the invention includes a rigid but somewhat flexible outer shell that has both an actuating area and a non-actuating area. These areas are designed so that the manual grasping force when applied to the actuating area electrically couples the inner conductive layer to the outer conductive layer, and when applied to the non-actuating area does not electrically couple the inner conductive layer to the outer conductive layer. This feature allows the operator to manipulate or adjust the power tool while grasping the non-actuating area to avoid undesirably toggling the tool off and on. A more elaborate embodiment of the invention features an inner channel that is generally cylindrical in form, and defines a hollow longitudinal space concentrically aligned within the inner channel. The hollow longitudinal space allows passage therethrough of a moving shaft or other energizeable element of the power tool.

To create the actuating and non-actuating areas on the outer shelf, the invention may include an inner channel having a proximal shelf and a distal shelf, and an outer conductive layer on the inner channel that extends between the proximal shelf and the distal shelf. Both the proximal shelf and the distal shelf are concentrically aligned with the inner channel, and one or both of the shelves has a diameter greater than a maximum width of the outer conductive layer. The difference between the greater diameter and the maximum width of the outer conductive layer defines a gap that electrically separates the inner conductive layer from the outer conductive layer. The non-actuating area lies above one of the two shelves, and the actuating area lies adjacent to the non-actuating area between the two shelves, where the grasping pressure can force the conductive layers into contact.

In another embodiment of the invention, a grasp-activated power switch serves as a handle of a hand-held power tool. The power switch includes an inner channel having an outer conductive layer, and an outer shell surrounding the inner channel, the outer shell having an inner conductive layer, an actuating area, and a non-actuating area. The power switch is configured such that a manual grasping force when applied to the actuating area electrically couples the inner conductive layer to the outer conductive layer, and when applied to the non-actuating area does not electrically couple the inner conductive layer to the outer conductive layer. The power switch may further include a battery and a wireless transmitter electrically coupled to the battery and to one of the inner conductive layer or the outer conductive layer, and configured for wireless coupling to a wireless receiver mounted elsewhere on the power tool. The power switch is configured so that the manual grasping force when applied to the actuating area causes transmission of power within the inner channel.

Another embodiment is a tattoo machine having a linear motor, a battery pack mounted on a proximal end of the machine and configured to energize the linear motor, and a shaft coupled to the linear motor and configured for attachment to a tattoo needle at the distal end of the machine, improved by a handle coupled between the proximal and distal ends of the machine, the handle including a grasp-activated power switch according to the present invention. The grasp-activated power switch includes an outer shell having an inner conductive layer, and an inner channel having an outer conductive layer and located within the outer shell so that the outer conductive layer is electrically separated from the inner conductive layer, wherein the inner channel defines a hollow longitudinal space for passage of the shaft therethrough. The grasp-activated power switch is configured so that a manual grasping force when applied to the outer shell electrically couples the inner conductive layer to the outer conductive layer to switch power off or on to the machine without obstructing movement of the shaft. A wireless transmitter is electrically coupled to one of the inner channel or the outer shell, and a battery is electrically coupled to another of the inner channel or the outer shell, so that the manual grasping force applied to the outer shell energizes the wireless transmitter by electrically coupling the wireless transmitter to the battery. The tattoo machine may further include a wireless receiver configured to receive a wireless signal transmitted by the wireless transmitter, and in response to receiving the wireless signal cause energization of the linear motor by the battery pack.

In yet another alternative embodiment, a grasp-activated power switch directly wired to an internal battery pack for a hand-held power tool is disclosed. By directly wiring the electrical connections internally, an outer shell or grip can be adjusted up and down to provide the maximum comfort to an operator. The adjustable grasp-activated power switch can be particularly useful for small precision hand-held power tools, such as a tattoo machine, where an operator may need to adjust the position of their grip on the tool to maintain precision in the working area. The grasp-activated power switch has an outer shell with an inner conductive layer electrically coupled to a first pin and to a second pin. The tool body within the outer shell has a first conductor and a second conductor, a battery, and a tool motor. The first and second pins extend inward toward the first and second conductors. The tool motor is electrically coupled to one of the first or second conductors and the battery is electrically coupled to the other conductor. Manual grasping force applied to the outer shell results in the first and second pin completing the electrical connection between the first and second conductors such that the tool motor is energized by the battery.

The outer shell is adjustable up and down the tool body between a maximum and minimum position. The first pin and second pin are positioned such that they will always contact the first conductor and second conductor, respectively, when the inner channel is attached between the maximum and minimum positions on the tool body. The inner channel may include threads for threaded engagement with tool body between the maximum and minimum positions.

In further embodiments according to the present invention, the grasp-activated power switch includes a switch body, an outer conductive shell substantially covering the switch body, and a tool body. The switch body includes an open-ended channel extending between a working end and a switch end. The channel defines an inner surface and an outer surface of the switch body. The inner surface includes an upper and a lower conductive bands which are electrically separated from one another. The outer surface includes an outer conductive surface and an outer conductive band, which are electrically separated from one another. The lower conductive band is electrically coupled to the outer conductive surface and the upper conductive band is electrically coupled to the outer conductive band. The tool body is electrically separated from the switch body and includes a first pin and a second pin. The distal end of the tool body adjustably engages the working end of the channel. The proximal end of the tool body includes a means for electrically coupling the tool body to a power source. The tool body also encloses a motor and a battery, which battery is electrically coupled to the motor. One of the pins is also electrically coupled to the battery and contacts the lower conductive band and the other of the pins is electrically coupled to the motor and contacts the upper conductive band. A manual grasping force applied to the outer conductive shell at the switch end causes the shell to complete the electrical connection thereby energizing the motor.

In some embodiments, the switch body includes a conductive pin extending through a sidewall to electrically couple the lower conductive band with the outer conductive surface. Similarly, a conductive strip may extend over a top end of the sidewall to electrically couple the upper conductive band with the outer conductive band. Alternatively, a second conductive pin may extend through the sidewall to electrically couple the upper conductive band with the outer conductive band. Preferably, the sidewall is nonconductive.

In preferred embodiments, each of the first pin and the second pin extending from the tool body are spring loaded. Preferably, the working end of the channel includes internal threads configured to engage complimentary external threads present on the distal end of the tool body. The internal threads of the channel define a length of the working end so that engagement of the distal end of the tool body along any length of the working end maintains contact between the first and second pins with the conductive bands formed about the inner surface of the channel.

Preferably, the battery included in the tool body is rechargeable. The power source coupling means preferably is electrically coupled to the motor. In some embodiments, the power source coupling means comprises a magnetic four pin connector configured to engage an external power source, such as a battery pack. The battery pack, engaged to the power source coupling means, is preferably configured to power the motor on and off in response to a pulse generated when the electrical connection is completed by the manual grasping force.

In further embodiments, the present invention relates to a power tool. The power tool has a tool body that encloses a motor, a grip removably attachable to the tool body, and a battery also removably attachable to the tool body. The grip has a grasp-activated switch. An illuminating filament partially or entirely circumvolves either the tool body or the battery. A manual grasping force applied to the grip causes the grasp-activated switch to electrically couple the battery to the motor and to the illuminating filament.

In more elaborate embodiments of the power tool, a tool body encloses a motor. The tool body has a distal end, a proximal end, and first and second pins extending outward from the tool body. A grasp-activated power switch has an inner body that is removably attachable to the distal end of the tool body. An outer conductive shell covers the inner body of the power switch. The inner body has a working end, a switch end and an open-ended channel extending between the working end and the switch end. The channel is configured to receive the distal end of the tool body. The channel has a defined inner and outer surfaces that are electrically coupled to one another. The inner surface is electrically separated from the first and second pins of the tool body. A battery is removably attachable to the proximal end of the tool body, and an illuminating filament at least partially circumvolves the tool body or the battery. The power tool is configured so that when a manual grasping force is applied to the grasp-activated power switch, the electrical connection between the first pin and the second pin is completed thereby energizing the motor and powering the illuminating filament.

### BRIEF DESCRIPTION OF THE DRAWINGS

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims. Component parts shown in the drawings are not necessarily to scale, and may be exaggerated to better illustrate the important features of the invention. Dimensions shown are exemplary only. In the drawings, like reference numerals may designate like parts throughout the different views, wherein:

FIG. 1 is a side view of one embodiment according to the invention of a grip for a hand-held power tool having an integral grasp-activated power switch.

FIG. 2 is a transparent and partially exploded side view of the grip of FIG. 1.

FIG. 3 is a partially cutaway side view of the grip of FIG. 1.

FIG. 4 is a distal end view of the grip of FIG. 1 with cap portion removed.

FIG. 5 is a perspective view of the cap portion of the grip of FIG. 1.

FIG. 6 is a perspective view of another embodiment according to the invention of a tattoo machine having an integral grasp-activated power switch.

FIG. 7 is a frontal view of the tattoo machine of FIG. 1.

FIG. 8 is an exploded frontal view of the tattoo machine of FIG. 1.

FIG. 9 is an exploded frontal view of the distal end of the tattoo machine of FIG. 1, showing an outer shell and inner channel.

FIG. 10 is a perspective view of the outer shell of a tattoo machine having an integral grasp-activated power switch according to the invention.

FIG. 11 is a distal end perspective view of an inner channel of a tattoo machine having an integral grasp-activated power switch according to the invention.

FIG. 12 is a proximal end view of an inner channel for one embodiment of a grasp-activated power switch according to the invention.

FIG. 13 is a perspective view of one embodiment of a battery pack for a tattoo machine having an integral grasp-activated power switch according to the invention.

FIG. 14 is a circuit diagram showing electrical connections in the grip area for one embodiment according to the invention for a hand-held power tool having an integral grasp-activated power switch.

FIG. 15 is a circuit diagram showing electrical connections in the battery pack area for one embodiment according to the invention for a hand-held power tool having an integral grasp-activated power switch.

FIG. 16 is a cross-sectional view of one embodiment according to the invention for a grasp-activated switch when a grasping force is applied to a non-actuating area.

FIG. 17 is a cross-sectional view of one embodiment according to the invention for a grasp-activated switch when a grasping force is applied to an actuating area.

FIG. 18 is an exploded transparent side view of an alternative embodiment according to the invention of a tattoo machine having an integral height adjustable grasp-activated power switch.

FIGS. 19A-19B are partially transparent side views of a tattoo machine showing the height adjustability of the height-adjustable grasp-activated power switch of FIG. 18

FIG. 20 is a circuit diagram showing electrical connections of the height adjustable grasp-activated power switch of FIG. 18.

FIG. 21 is a partially exploded side view of an alternative embodiment of a grasp-activated switch according to the present invention deployed with a tattoo machine.

FIG. 22 is an exploded side view of a grasp-activated switch according to the embodiment shown in FIG. 21.

FIG. 23 is an end view of an embodiment of a switch body and outer shell according to the embodiment shown in FIG. 21.

FIG. 24 is a side cross-sectional view taken along lines A-A marked in FIG. 23 of the switch body and outer shell according to the present invention.

FIG. 25 is a circuit diagram showing electrical connections of the grasp-activated power switch of FIG. 21.

FIG. 26 is a partially transparent side view of an embodiment of a grasp-activated power switch according to the present invention.

FIG. 27A and FIG. 27B are cross-sectional side views, taken along lines B-B marked in FIG. 26, demonstrating a grasping force applied to complete the electrical connection according to the present invention.

FIG. 28 is approximal end view of an embodiment of a tool body according to the present invention.

FIG. 29 is a distal end view of an embodiment of a battery pack deployable with the grasp-activated power switch shown in FIG. 21.

FIG. 30 is an exploded side view of an alternative embodiment of a power tool according to the present invention.

FIG. 31 is an exploded side view of a further alternative embodiment of a power tool according to the present invention.

FIG. 32 is a circuit diagram of a control scheme for a power tool according to the invention as depicted in FIG. 30 or FIG. 31.

FIG. 33 is a perspective view of the embodiment shown in FIG. 30 changing state.

FIG. 34 is a perspective view of the embodiment shown in FIG. 31 changing state.

FIG. 35 is a perspective view of the embodiment shown in FIG. 33 in use.

FIG. 36 is a perspective view of the embodiment shown in FIG. 34 in use.

### DETAILED DESCRIPTION OF THE INVENTION

The following disclosure presents exemplary embodiments for systems and methods that employ an integral grasp-activated power switch for a hand-held power tool according to the present invention. The grasp-activated power switch allows an operator of the tool to turn the tool on and off by squeezing the grip once to turn on and again to turn off, in toggling fashion. As used throughout this disclosure, the term “grasp-activated” means causing the operation of an electrical switch by a squeezing or clamping action of the operator's hand on a handle or grip portion of the power tool being switched. A grasp-activated action is therefore not accomplished by pushing a button, turning a dial, or by twisting, pushing, or pulling a lever. A grasp-activated action is accomplished by forcing together two opposing sides of a grip or handle by an operator squeezing the grip or handle between his thumb and forefinger.

The present invention applied to tattoo machines improves the comfort, posture, and ease of tattooing for the tattoo artist, and also improves the means for actuating the tattoo machine power supply. The invention allows the artist's operating hand (i.e. the hand that holds the machine) to activate the battery by turning it on and off with a squeeze of the grip for a more seamless tattooing experience. The principles of the present invention may have utility in applications other than tattoo machines, for example, in hand-held power tools such as medical instruments, dental instruments, high-speed drills, etc., especially wherever the tool operator performs high-precision work that requires frequent stopping and restarting of the power tool.

The invention gives tattoo artists the ability to control the power switch of the tattoo machine very easily, when necessary to stop and grab ink, paper towels, or other accessories. The invention is particularly useful when an artist suddenly becomes aware that a supply of ink in an ink cap has run low, because it allows the artist to very easily stop the tattoo machine to prevent the needle from impacting the bottom of the ink cap. Also, about every hour or so, a tattoo artist needs to stop work, stand up, and stretch and with the present invention this can be more easily done. Enhanced power control provided by the grasp-activated grip of the present invention allows the artist to minimize battery power consumption, minimize the likelihood of contaminating the needle, and minimize the risk of accidental injury by puncture from a tattoo needle.

According to the invention, actuation of the grasp-activated switch can connect the tattoo machine motor to the main battery either by directly-coupled wire, or wirelessly by means of an RF or Bluetooth™ transmitter coupled to the grasp-activated switch. Such a wireless transmitter is configured to communicate with a complementary wireless receiver that is coupled to the main battery. The present invention may be integrated into a grip alone, for removable installation on existing tattoo machines or other tools, or the invention may be integrated as part of an entire hand-held power tool system.

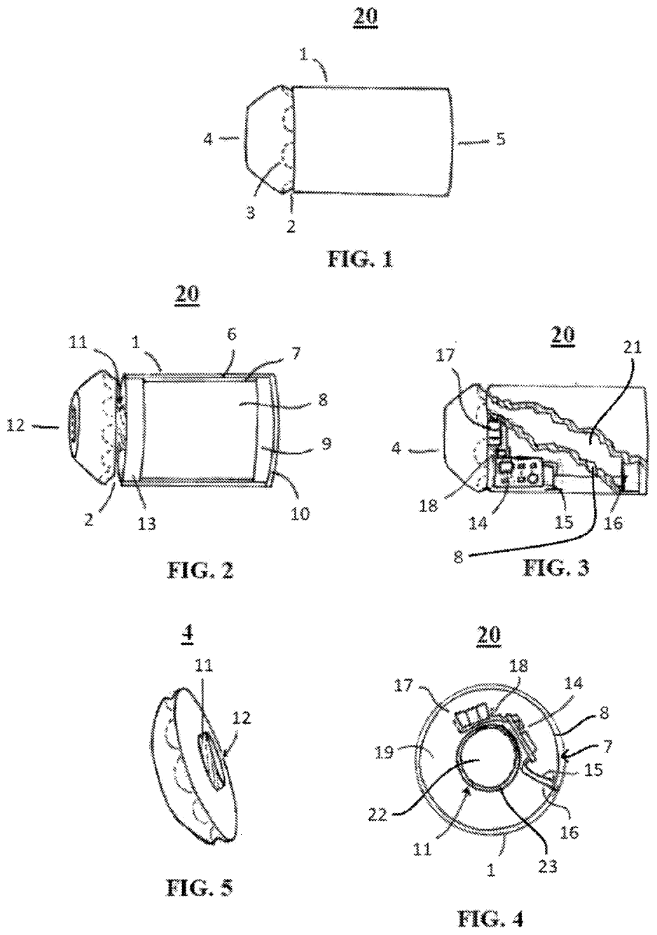

FIG. 1 shows a side view of one embodiment according to the invention of a grip 20 for a hand-held power tool having an integral grasp-activated power switch. The grip 20 includes an outer thin plastic wall or shell 1 that provides a gripping surface that allows an operator to manually grasp and control the tattoo machine during operation. A seam 2 is defined between the distal end of the outer shell 1 and a distal end cap 4. The distal end cap 4 is removably attachable to the distal end of shell 1, and may include one or more grooves, protrusions, or indentations 3 that are formed on an outer surface of the end cap 4, as in the example shown, to enable the operator to more easily manually attach or detach the end cap 4 to or from the outer shell 1. An opening 5 at the proximal end of the outer shell 1 provides a means for connecting the grip to a motive force and power supply for energizing the tool.

The outer shell 1 has dual-purpose utility: first, it provides a gripping surface to allow an operator to grasp the tool firmly by one hand, i.e. between thumb and forefinger, while directing and manipulating the tool; and second, it provides a manually operable, or grasp-activated, switch that allows the operator to switch power on and off to the power tool while grasping the tool. In particular, the outer shell 1 is configured with both a non-actuating area and an actuating area. In the embodiments illustrated herein, the non-actuating area occurs at the distal end of the outer shell, and the actuating area occurs away from the distal end of the outer shell, at or near the middle portion of the outer shell, for example. This configuration allows the operator to grasp the outer shell at the non-actuating area and operate the tool without causing the tool to switch on or off, or slightly adjust his grip and grasp the outer shell at the actuating area where he can switch power to the tool on or off by gently squeezing the outer shell 1 between his thumb and forefinger.

To enable the dual-purpose utility of the outer shell 1, the outer shell 1 is preferably formed from a rigid but slightly resilient material such as a thermoplastic—ABS, nylon, polycarbonate, polyethylene, etc.—or from a thin metal sheet such as steel or aluminum that will allow for slight flexing. In another embodiment, a hard synthetic rubber may be used to form the outer shell. When used for tattoo machines or on medical or dental instruments, an advantage provided by the invention is the option to design the outer shell 1 to be disposable, for sanitary purposes. Thus, longevity of service life may not be an essential design basis, so that less expensive materials of construction can be selected for the outer shell.

FIG. 2 shows a transparent and partially exploded side view of the grip 20. In this view, the distal end cap 4 is shown partially detached from the grip 20 to reveal threads 11 that illustrate one possible means for removably attaching the distal end cap 4 to the grip 20. The transparent view reveals that the outer shell 1 has an inner conductive layer 6 disposed onto its inner surface. In the embodiment shown herein, the outer shell 1 is generally cylindrical, and the inner conductive layer 6 also forms a generally cylindrical inner wall. The inner conductive layer may be the inner surface of an outer shell 1 that is composed of metal. Or the inner conductive layer 6 may be a thin metal sheet material curved into a cylindrical form and attached to a non-metallic inner surface of the outer shell 1, or the inner conductive layer 6 may be a pre-formed cylindrical metal pipe, or it may be a portion of a conductive metal sheet, pipe, or other material attached to a non-conductive material that forms part or all of the outer shell 1.

The grip 20 also includes an inner channel 8 that is concentrically aligned with the outer shell 1. A hollow longitudinal space 22 runs through the center of the inner channel 8 from proximal opening 5 to distal opening 12. The inner channel 8 may also be generally cylindrical in form, and has an outer conductive layer 21 on its outer surface that is displaced from the inner conductive layer 6 by a gap 7. Gap 7 may be on the order of about 0.036 inches in width, although greater and lesser such widths are certainly possible within the scope of the invention. And while the embodiment of grip 20 disclosed herein has a generally cylindrical form, with generally cylindrical conductive layers 6 and 8, other geometric configurations are possible within the scope of the invention that maintain a gap 7 of a desired width. The desired width of the gap 7 is a distance sufficient to prevent electrical current flow across the gap when the grip is not being squeezed when the tool is electrically energized, but that also provides good electrical contact between conductive layers 6 and 21 when the actuating area of outer shell 1 of the electrically energized tool is being squeezed with manual force.

To maintain the form and integrity of the grip 20, the inner channel 8 is formed from a rigid material that is generally not flexible and will not deform under a manual gripping force. Materials such as metal or hard plastic are suitable for forming the inner channel 8. To create the non-actuating and actuating areas of the outer shell 1, a proximal shelf 9 and a distal shelf 13 are formed on respective ends of the inner channel 8. The shelves 9 and 13 are similarly formed from rigid materials, and may be integral to the inner channel 8, or rigidly attached thereto. The outer shell 1 spans over both the proximal shelf 9 and the distal shelf 13. Preferably, shelves 9 and 13 are made from a dielectric material, with one or both of the shelves 9 or 13 having a conductive outer surface. In the embodiments shown herein, the proximal shelf 9 has a dielectric surface and the distal shelf 13 has a conductive surface. When the grip 20 is in the “shelf” position, that is, resting on a shelf or otherwise in a condition not being grasped or squeezed by an operator's hand, the conductive surface of the distal shelf 13 is electrically separated from the outer conductive layer 21 on the inner channel 8. Also while in the shelf position, the inner conductive layer 6 of the outer shell 1 forms a conductive bridge over shelves 9 and 13. In the shelf position, the inner conductive layer 6 may contact the conductive surface of the distal shelf 13 and the dielectric surface of shelf 9, but does not contact the outer conductive layer 21, being separated therefrom by the gap 7. The non-actuating area of the outer shell 1 therefore occurs at or near the distal end of the outer shell, where pressure between the operator's thumb and forefinger will force the inner conductive layer 6 against the conductive surface of the distal shelf 13, but not deform the outer shell 1 to cause any part of the inner conductive layer 6 to close the gap 7 and make contact with the outer conductive layer 21 of the inner channel 8. Accordingly, the actuating area of the outer shell 1 occurs a short distance away from the distal end of the outer shell 1 at an intermediate location between the proximal shelf 9 and distal shelf 13, where pressure between the operator's thumb and forefinger will slightly deform the outer shell 1 and force the inner conductive layer 6 into electrical contact with the outer conductive layer 21 of the inner channel 8, thereby closing the grasp-activated switch.

The inner channel 8 may include complementary means for removably receiving the distal end cap 4. In this example, such receiving means comprises female threads formed on the distal end of the inner channel 8 and sized to engage with male threads on the distal end cap 4. An opening 12 is defined through the distal end cap 4, to accommodate the distal end or working element of the power tool, e.g., a saw blade, drill bit, or the needle end of a tattoo machine. Opening 12 passes all the way through the longitudinal center of grip 20 to the proximal opening 5 to allow the grip 20 to be installed on the power tool and to allow the working element at the distal end of grip 20 to be coupled to the motor of the power tool at the proximal end of grip 20.

FIG. 3 shows a partially cutaway side view of the grip 20. This view reveals electronic components that are mounted to the inner channel 8 and that make up a portions of the electrical circuit of the grasp-activated power switch. These components include a circuit board, a battery 17, and wiring 15, 16, 18. The circuit board includes an RF transmitter 14. The battery 17 provides power to the transmitter 14. Battery 17 may consist of multiple batteries ganged in series and/or parallel to achieve a desired voltage. Each such battery may be lithium ion type or another known type of battery technology, disposable or rechargeable. In one example, battery 17 consists of a single 3.7-V lithium ion disposable battery. In another example, battery 17 consists of three 1.5-V batteries.

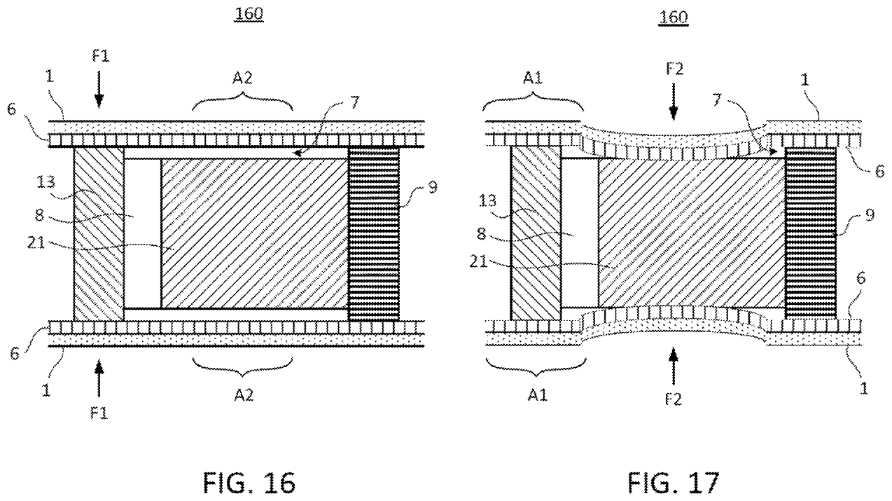

In FIG. 3, the battery 17 is mounted to the distal shelf 13. In other embodiments, the battery 17 may be mounted to the distal end cap 4, to the outer shell 1, or to another location on grip 20. Wiring 15 electrically couples the positive terminal of the transmitter 14 to the outer conductive layer 21 of the inner channel 8. Wiring 16 electrically couples the positive terminal of battery 17 to the inner conductive layer 6 of the outer shell 1. Wiring 18 connects the negative terminal of the transmitter 14 to the negative terminal of battery 17 to complete the circuit, so that when a sufficient manual grasping force squeezes grip 20 to make electrical contact between the inner conductive layer 6 and the outer conductive layer 21, the grasp-activated switch closes and as a result transmitter 14 is energized by the battery 17. See FIG. 16.

When the transmitter 14 is energized, it transmits an on/off signal or pulse to a complementary receiver 24 that is mounted on or near the main power supply for energizing the motor of the hand-held tool. For example, where the hand-held tool is a tattoo machine, the main power supply may be a main battery pack mounted at the proximal end of the machine. In this way the main battery back is configured for wireless communication with the transmitter 14. When the receiver 24 receives the on/off signal, it causes a switch at the output of the main battery pack to change state, using any of numerous flip-flop type logic circuits well known in the electrical engineering arts. That is, if the main battery pack switch was off when the on/off signal is received, it changes state to on, and if the battery pack switch was on when the on/off signal is received, it changes state to off. In this manner an operator of the hand-held tool using a grip 20 according to the present invention can toggle power off and on to the motor of the tool by momentarily squeezing the actuating area of outer shell 1 with his operating hand that is already grasping the grip 20.

One advantage of using the combination wireless transmitter 14 and receiver 24, rather than hard-wiring the grasp-activated switch to the terminals of main power supply, is to provide a disposable part that can be easily installed onto the hand-held tool with no effect on the wiring. For example, after a tattooing session, the operator can disconnect the end cap 4, pull the grip 20 (consisting of the shell 1 and inner channel 8) off the distal end of the tool, and discard the grip 20 as medical waste. Another advantage of the wireless switch is to avoid having to run wiring in close proximity to moving parts of the tool, which is especially useful when using the grasp-activated switch of the present invention as an after-market modification to a commercially available tool. In other embodiments of the invention, however, the grasp-activated switch may be hard-wired to the main power supply of the tool, to reduce the overall manufacturing cost of the grip, and possibly increase reliability by eliminating the need for the battery 17.

FIG. 4 shows a distal end view of the grip 20 with distal end cap 4 removed. This view shows wiring 18 that couples the negative terminal of battery 17 to the negative node of the transmitter 14. In one embodiment, the grip 20 may include a dielectric core 19 between the inner channel 8 and the hollow longitudinal space 22 that runs through the center of the inner channel 18 from proximal opening 5 to distal opening 12. The dielectric core 19 adds strength and rigidity to the grip, and may be made from a resilient material to assist the grip in restoring itself to original form when an operator's grasping force is released. In one embodiment, the dielectric core 19 may be formed from an epoxy. In another embodiment, the dielectric core 19 may comprise silicone. In another embodiment, the dielectric core may provide structural support and insulation to one or more electrical components of the grip 20. In one example, the transmitter 14, battery 17, and associated wiring and/or circuit boards may be embedded within the material of the dielectric core during manufacture while the core is in liquid or gel form prior to setting.

The hollow longitudinal space 22 runs through an interior wall 23 that is concentrically aligned with the longitudinal axis of the inner channel 8. Space 22 is configured to allow the working end of the hand-held tool to pass through the grip 20 unobstructed. The interior wall 23 is formed from a rigid material configured to engage one or more bearings or other components that support a shaft of the motor of the hand-held tool. The interior wall 23 has sufficient strength to resist deformation when an operator squeezes the outer shell 1, so that tool operation is unobstructed during the transition of power from off to on or vice versa.

FIG. 5 shows a perspective view of the distal end cap 4 of the grip 20, to illustrate threads 11 that surround the opening 12. The threads 11 may be configured to engage complementary threads formed on the distal end of the interior wall 23. The end cap 4 may assume a variety of shapes, depending on the configuration of the working end of the tool. Ideally, when the end cap 4 is fully engaged to the interior wall 23, a tight seal is formed at seam 2 to insulate the electronic components from contamination.

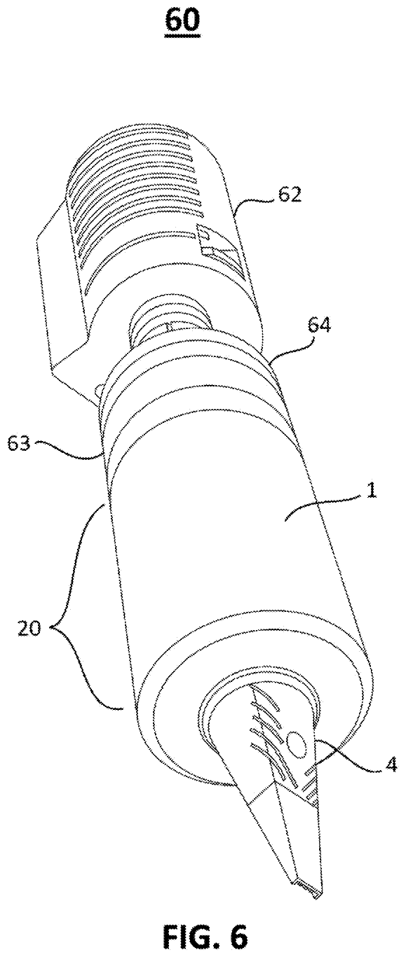

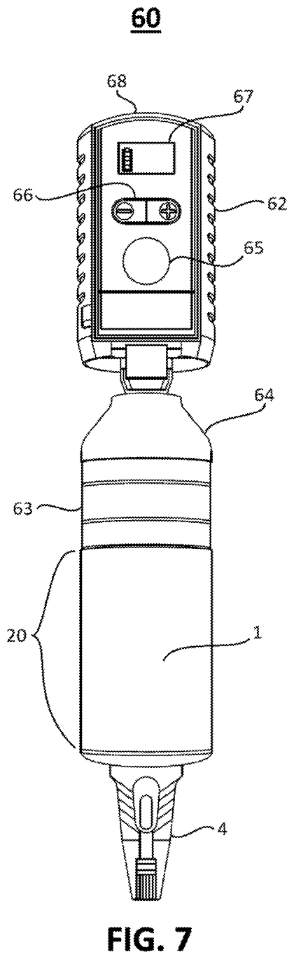

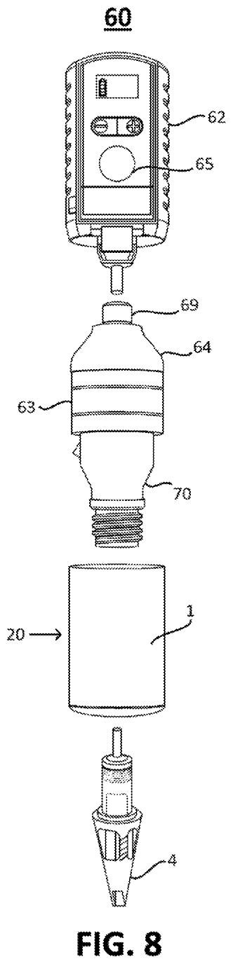

FIG. 6 shows a perspective view of another embodiment according to the invention. In this embodiment, the hand-held tool that bears the grasp-activated power switch is a tattoo machine 60. Tattoo machine 60 is a commercially available machine that is modified by installing a grip 20 of the present invention as an after-market modification. An outer shell 1 of the grip 20 is shown in the figure. A battery compartment 62, also modified according to the invention, is connected to the proximal end of the tool 60. An end cap 4 is attached to the distal end of the tool 60, to protect a reciprocating tattoo needle driven by a linear motor that is enclosed within a motor casing 64. A grip 63, provided by the original tattoo machine manufacturer, allows a user to grasp the machine 60 while operating it; however, grip 63 is a static device that does not provide a grasp-activated switch. FIG. 7 shows a frontal view of the tattoo machine 60. Several additional optional features are shown on the battery compartment 62. These include a manual on/off pushbutton 65, a voltage up/down toggle switch 66, an LED display 67, and a lid 68. FIG. 8 shows an exploded frontal view of the tattoo machine 60. This view reveals the proximal end of the linear motor 69 that lies within the motor casing 64, and also the distal end 70 of the machine as provided by the original tattoo machine manufacturer.

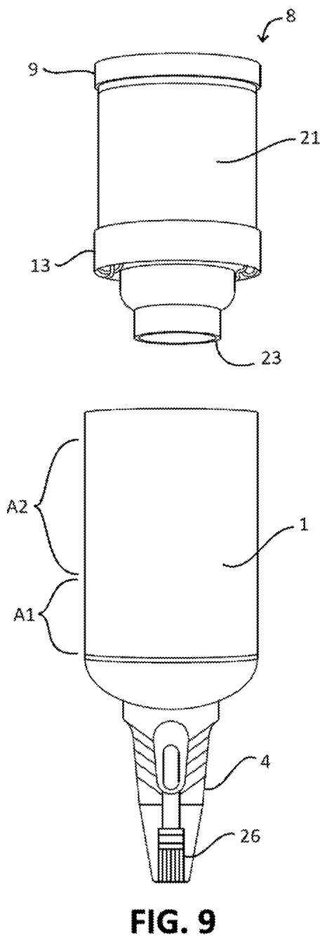

FIG. 9 shows an exploded frontal view of the distal end of the tattoo machine 60. This view illustrates two main components of the invention that are used in the modification of machine 60—the outer shell 1 and the inner channel 8. Threading on interior wall 23 may be used by an operator to adjust the length of protrusion of a tool element, such as a tattoo needle 26, beyond the distal end of the end cap 4. While making this adjustment, with the machine fully assembled as shown in FIG. 7, the operator may grasp the outer shell 1 of grip 20 at its non-actuating area A1 and press the outer shell 1 against the distal shelf 13, then rotate the end cap 4 with respect to the grip 20. In this manner, the adjustment can be made without switching the machine on or off.

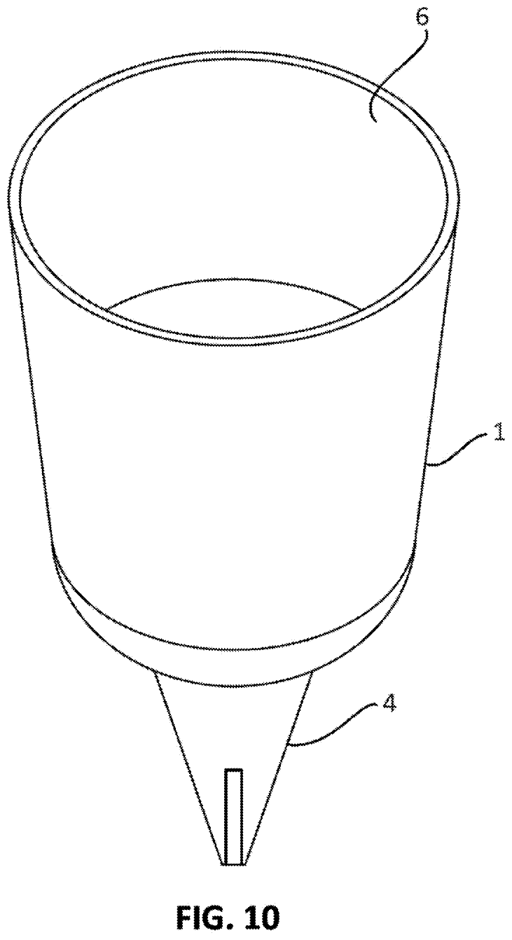

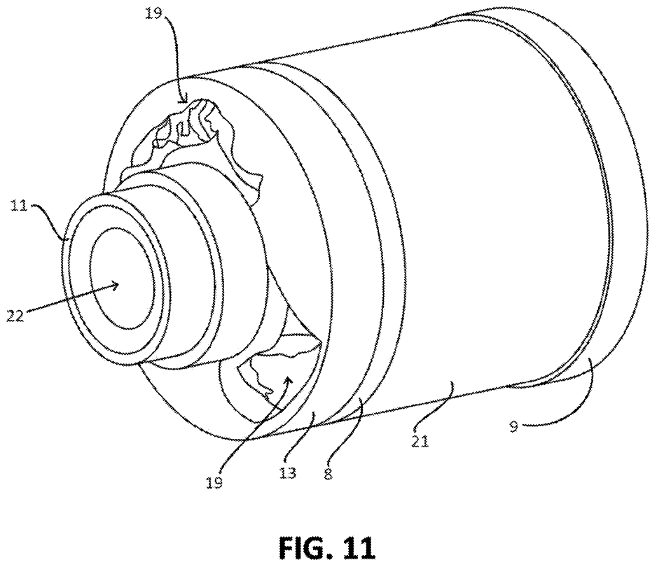

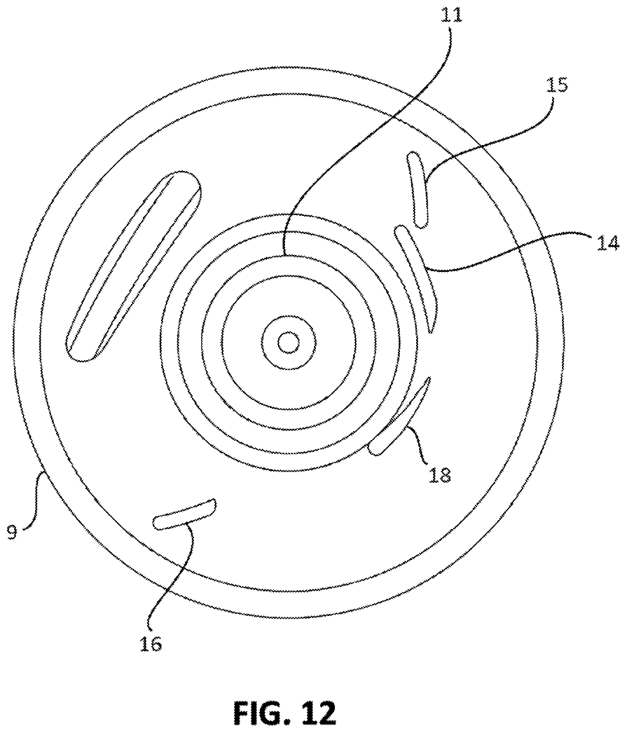

FIG. 10 shows a perspective view of the outer shell 1 of the tattoo machine 60. The inner conductive layer 6 is shown on the inner surface of the outer shell 1. FIG. 11 shows a distal end perspective view of an inner channel of a tattoo machine having an integral grasp-activated power switch according to the invention. The dielectric core 19 made from epoxy or silicone fills the void between inner channel 8 and the hollow longitudinal space 22. Various electronic components and wiring may be embedded within the core 19. FIG. 12 shows a proximal end view of an embodiment of an inner channel 8.

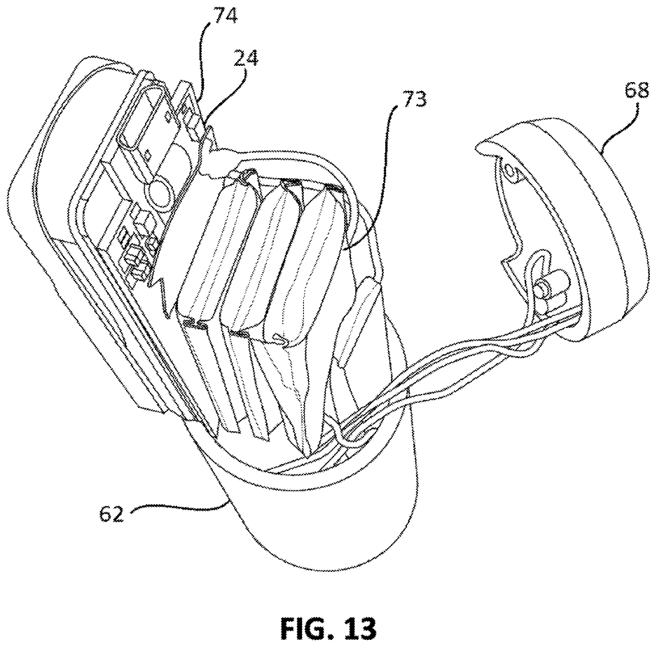

FIG. 13 is a perspective view of one embodiment of a battery pack 62 for a tattoo machine having an integral grasp-activated power switch according to the invention. Battery pack 62 includes one or more individual batteries 73 arranged as a pack in a series or parallel configuration to achieve a desired voltage needed to drive the machine's DC motor, usually 7.5V to 8.5V for a tattoo machine. The manual on/off switch 65 is bypassed so that the on/off switching operation can be performed by an electric circuit provided on a circuit board 74 that is mounted within the battery pack 62. The circuit board 24 includes a receiver 24 that is configured to receive and respond to an RF signal from the transmitter 14 that is mounted to the grasp-activated switch 20. Other components on circuit board 24 are shown in the circuit diagram of FIG. 15.

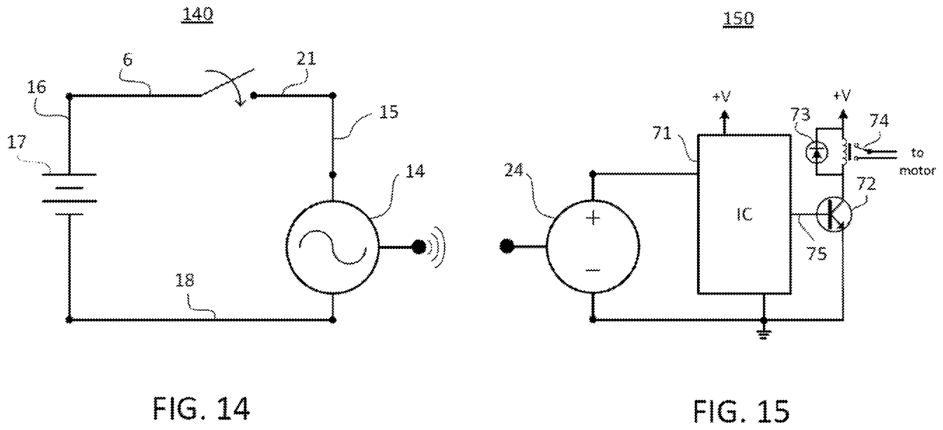

FIG. 14 is a circuit diagram 160 that shows typical electrical connections in the grip area for one embodiment according to the invention for a hand-held power tool, such as a tattoo machine, having an integral grasp-activated power switch. Wiring 16 connects the positive terminal of battery 17 to the inner conductive layer 6. The outer conductive layer 21 is connected by wiring 15 to the positive terminal of the RF transmitter 14. The negative terminal of transmitter 14 is connected by wiring 18 to the negative terminal of battery 17. When an operator squeezes the actuating area of the outer shell 1, the inner conductive layer 6 is pressed into electrical contact with the outer conductive layer 21 to close the switch and complete the circuit. This action energizes the transmitter 14, which sends a momentary RF pulse wirelessly to receiver 24 that is mounted in the battery pack on the proximal end of the tool. The operator need only squeeze the grip momentarily to switch the power on or off to the tool. A subsequent squeeze will cause the tool to change state again, either from on to off or off to on.

FIG. 15 is a circuit diagram showing electrical connections in the battery pack area for one embodiment of a hand-held power tool, such as a tattoo machine, having an integral grasp-activated power switch. The RF receiver 24 is configured to receive and respond to the RF pulse transmitted by transmitter 14. The response of the receiver 24 is to generate a pulse, such as a voltage spike of about 5 volts, across its terminals using methods known in the art. The pulse is fed to an input pin of an integrated circuit (IC) 71 that is configured as a toggle switch flip-flop. For example, an IC 4017 may be used for this purpose, by connecting pin #4 to pin #15 so that each successive pulse at the input toggles the output between 0 and +5V. The toggling output pin is coupled to the base 75 of a transistor 72 configured to behave as a solid state switch. Transistor 72 will therefore change state with each successive pulse, alternately enabling and disabling current flow to a relay 74 that is magnetically coupled to a normally open switch. When current flows through transistor 72, relay 74 is energized, closing the switch that provides power from batteries 72 to the tool motor, such as linear motor 69 described in a previous embodiment. When current through transistor 72 is cut off, relay 74 de-energizes and the switch opens to stop the motor. Circuits 140 and 150 can thereby perform the same function as the manual on/off pushbutton switch 65, to allow an operator to toggle the tool on and off. But unlike switch 65, an operator using the present invention can toggle the power by momentarily squeezing the grip.

FIGS. 16 and 17 are a cross-sectional views of one embodiment according to the invention for a grasp-activated switch 160. Switch 160 is similar in form and function to the grasp-activated switch described within grip 20 as shown in FIGS. 1-5. FIG. 16 illustrates the configuration of switch 160 when a grasping force F1 is applied to a non-actuating area A1. FIG. 17 illustrates the configuration of switch 160 when a grasping force F2 is applied to an actuating area A2. In general, the non-actuating area A1 of the switch occurs at the distal end of the assembly on and about the area where the outer shell 1 covers the distal shelf 13. In one embodiment, the non-actuating area occurs approximately along the “distal third” of the grip. The actuating area A2 of the switch occurs along an area proximally adjacent to area A1, along the middle area of the outer shell 1. In one embodiment, the actuating area A2 occurs along about twice the length of the non-actuating area A1. In other embodiments, the size, length, and location of areas A1 and A2 may vary, depending on tool type, size and other design considerations. In another example, the proximal shelf 9 may be made conductive and separated from the outer conductive layer 21, and the distal shelf 13 may be made non-conductive, so that the distal-to-proximal locations of areas A1 and A2 are reversed, with A2 located on the distal side of A1.

In FIG. 16, a grasping force F1 is applied to the non-actuating area A1. This action has no effect on the grasp-activated switch, and so the switch remains open, with gap 7 separating inner conductive layer 6 from outer conductive layer 21. An operator may grasp the outer shell 1 in this manner while operating or adjusting the tool.

In FIG. 17, the operator changes the state of the grasp-activated switch by momentarily adjusting his grip on the outer shell 1 to place his thumb and forefinger within the actuating area A2 and squeezing the outer shell 1 with a quick, firm pulse. This action drives the inner conductive layer 6 across the gap 7 into electrical contact with the outer conductive layer 21, thereby completing the circuit, e.g. as shown by the switch in circuit 140. As described in the context of circuits 140 and 150, the grasping pulse of the operator at area A2 causes a change of state in the operation of the power tool, from on to off or from off to on.

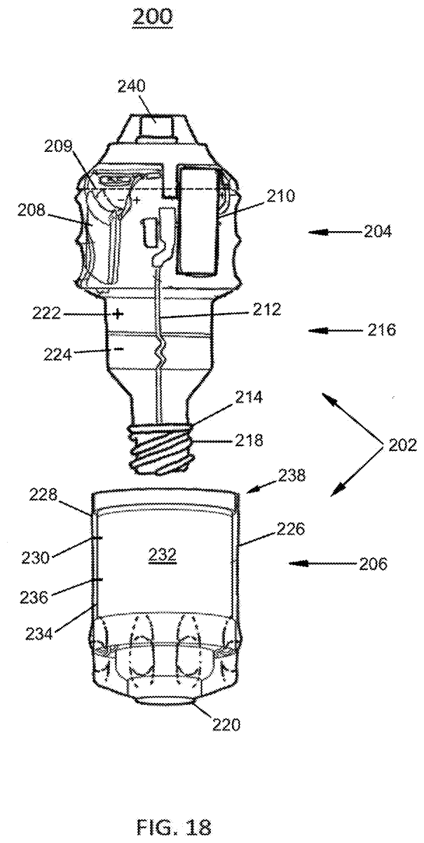

FIG. 18 is an exploded transparent view of an alternate embodiment according to the present invention of a tattoo machine 200 having a height-adjustable grasp-activated power switch 202. The tattoo machine 200 has a tool body 204 and an attachable grip 206. The tool body encloses a battery 208 electrically coupled to a tool motor 210. (Note, the dotted line at 209 is meant to demonstrate a wired connection between the battery 208 and the tool motor 210.) A power attachment end 240 of the tool body 204 is configured to receive an external battery pack 62 or be wired directly into a power source, e.g., a conventional cord and plug. Connection between the power attachment end 240 of the tool body 204 and the external battery pack 62 can be accomplished through conventional means. The external battery pack 62 is one source of power for the tool motor 210 of the present embodiment. Alternatively, a convention cord and plug (not shown) can be used to provide power to the tool motor 210. The tool motor is mechanically coupled to a tattoo needle plunger 212 configured to deliver tattooing ink to a working surface, e.g., the surface of the skin being tattooed. The distal end 214 of the tool body 204 is configured to removably attach the grip 206 so that an actuating area 216 of the tool body is completely encompassed by the grip, which position can be adjusted up and down the actuating area. In preferred embodiments, the distal end 214 of the tool body 204 includes male threads 218 configured for threaded engagement with corresponding female threads positioned within the distal end 220 of the grip 206.

The tool body 204 also has a first conductor 222 and a second conductor 224 that are formed as integral parts of the actuating area 216 of the tool body 204. The first conductor 222 is electrically separated from the second conductor 224. The tool motor 210 is electrically coupled to either the first conductor 222 or the second conductor 224 and to the battery 208. The other of the first conductor 222 or the second conductor 224 is electrically coupled to the battery 208 to form an open circuit. The grip 206 has an outer shell 226 that is electrically separated from the tool body 204. The outer shell 226 has an inner conductive layer 228 that is electrically coupled to a first pin 230 and to a second pin 236. The first pin 230 and the second pin 236 extend inward towards the first conductor 222 and second conductor 224 on the tool body 204. The outer shell 226 has a preferably hollow internal channel 232. The first pin 230 and the second pin 236 are configured to complete the electrical connection between the first conductor 222 and the second conductor 224 when a manual grasping force is applied to the actuating area 216 when the grip 206 is engaged to the tool body 204. This energizes the tool motor 210. To de-energize the tool motor 210 when it is desired to shut off the tattoo machine 200, an operator reapplies the grasping force to the actuating area 216.

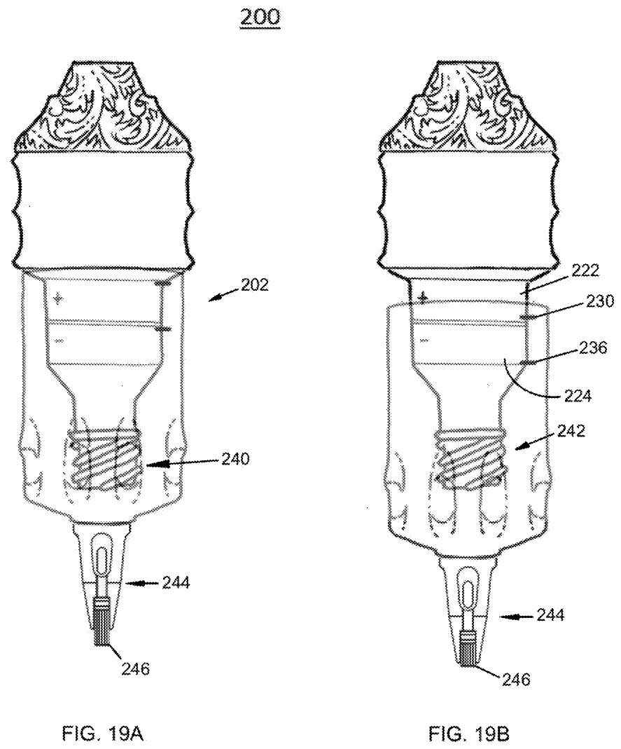

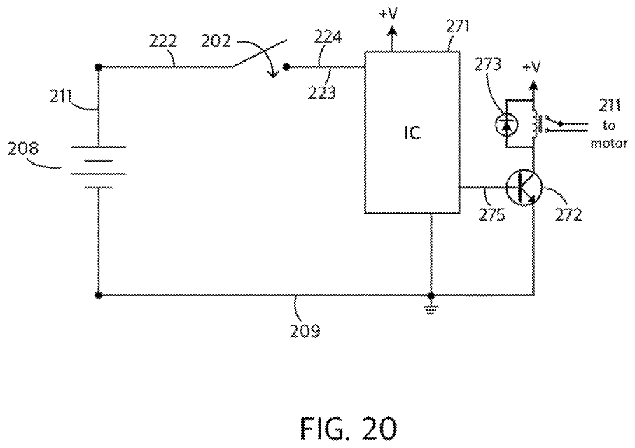

FIGS. 19A and 19B are partially transparent views of the tattoo machine 200 demonstrating the adjustable positioning of the grasp-activated power switch 202 on actuating area 216 of the tool body 204. A needle cap 244 is attached to the distal end 220 of the grip 206 and provides cover for a tattoo needle 246 that is coupled to the needle plunger 212. By adjusting the grip 206 up or down relative to the tool body 204, an operator can now readily control the length of the tattoo needle 246 protruding out of the needle cap 244 which defines the depth the needle 246 can penetrate the working surface, e.g., surface of the skin being tattooed. Depth of penetration for a tattoo needle is an important feature that needs to be controlled by the tattoo artist Different tattooing methods require different needle penetration depths. Further, regardless of the method, a tattoo artist will be required to periodically adjust the needle penetration depth based on which part of the body the artist is currently tattooing, e.g., bony areas where the skin is tight versus more fatty/muscular areas where the skin is looser. To achieve this adjustability, the actuating area 216 has a maximum attachment position 240 for attaching the grip 206 to the tool body 204 and a minimum attachment position 242. The grip 206 is designed so that the first pin 230 and the second pin 236 maintain the ability to contact the first conductor 222 and the second conductor 224 when the grip 206 is attached to the tool body 204 between the maximum attachment position 240 and the minimum attachment position 242 of the actuating area 216. In the illustrated embodiment, the maximum attachment position 240 is achieved when the female threads at the distal end 220 of the grip 206 are fully engaged with the male threads 218 at the distal end 214 of the tool body 204. The minimum attachment position 242 is achieved when the female threads at distal end 220 are partially engaged with the male threads 218. The partial threaded connection positions the grip 206 a distance forward from the tool body 204 such that the needle cap 244 covers more of the needle 246 thereby decreasing the depth of penetration.

FIG. 20 is a circuit diagram showing electrical connections for the tattoo machine 200 of FIGS. 18-19B. Wiring 211 connects the positive terminal of the battery 208 to the first conductor 222. The second conductor 224 is connected by wiring 223 to the positive terminal of the integrated circuit (IC) 271. The integrated circuit 271 can be similar to integrated circuit 71 described above, i.e., configured as a toggle switch flip-flop. Wiring 209 grounds the negative terminal of the integrated circuit 271 with the negative terminal of the battery 208. When the power switch 202 is grasped by an operator, the first and second pins 230, 236 contact the first conductor 222 and second conductor 224 to close the circuit generating a pulse that is fed as an input signal to the integrated circuit 271 which toggles the output between, for example, 0 and +5V. The toggling output pin is coupled to the base 275 of a transistor 272 configured to behave as a solid state switch. Transistor 272 thus changes state with each successive pulse, alternately enabling and disabling current flow to a relay 274 that is magnetically coupled to a normally open switch. When current flows through transistor 272, relay 274 is energized, closing the switch that provides power from batteries 273 to the tool motor 210. When current through the transistor 272 is cut off, relay 274 de-energizes and the switch opens to stop the motor. The batteries 273 can an external, rechargeable battery pack, similar to the battery pack 62 previously described.

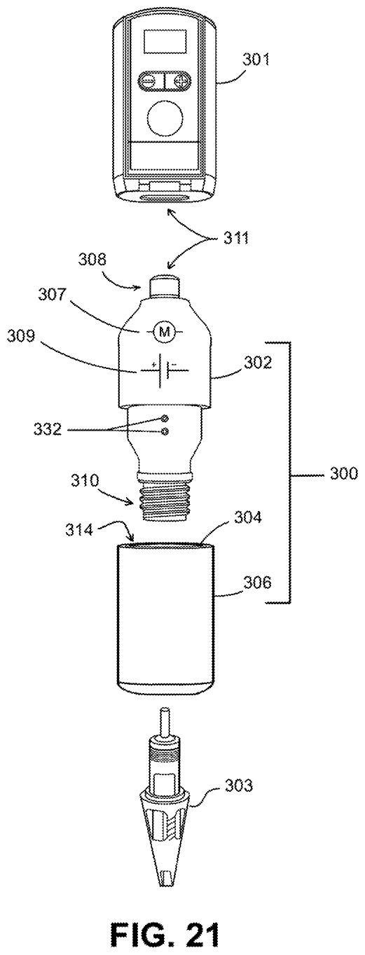

FIG. 21 is an exploded side view of an alternative embodiment of a grasp-activated power switch engineered for a tattoo machine. The power switch 300 includes a tool body 302, a switch body 304 and an outer conductive shell 306 enclosing at least a portion of the switch body 304. The switch body 304 includes an open-ended channel 314 extending therethrough. Note, in this view, the outer conductive shell 306 encloses the switch body 304 so that the two components appear as one part. When used with a tattoo machine as shown in FIG. 21, an external battery pack 301 engages the proximal end 308 of the tool body 302 and a tattoo needle cartridge 303 engages the distal end 310 of the tool body 302, as will be detailed further below. The proximal end 308 has a means for electrically coupling to a power source, such as the external battery pack 301 illustrated. The power source coupling means 311 of the proximal end 308 of the tool body 302 will be detailed further below.

When assembled, the tool body 302 engages the switch body 304 through the open-ended channel 314. The tool body 302 is electrically separated from the inner surface 316 of the open-ended channel 314. The tool body 302 encloses a motor 307 and a battery 309 between the proximal end 308 and distal end 310 thereof. Two pins 332 extend from the outer surface of the tool body 302. The battery 309 is electrically coupled to the motor 307 and one of the two pins 332 whereas the motor 307 is electrically coupled to the other of the two pins 332. The two pins 332 extend toward the inner surface 316 of the switch body 304, as will detailed further below. In preferred embodiments, the two pins 332 are each spring loaded and biased toward the inner surface 316.

In some embodiments where the power switch 300 is configured for a tattoo machine, the motor 307 is mechanically coupled to a linear shaft configured to engage the tattoo needle cartridge 303, as is known in the relevant art. In such embodiments, when the motor 307 receives power, the linear shaft moves the tattoo needle cartridge 303, as is conventional for tattoo machines.

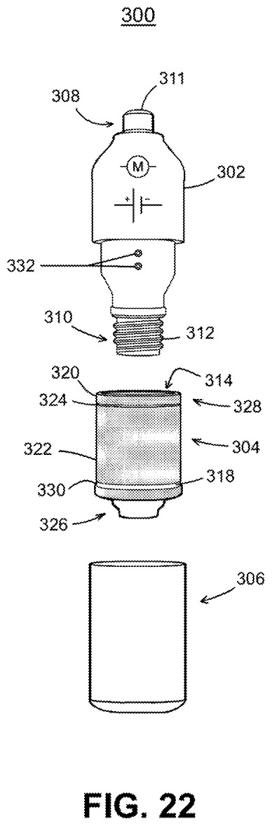

FIG. 22 is an exploded side view of an embodiment of the power switch 300. The distal end 310 of the tool body 302 preferably includes external threads 312 configured to engage complimentary threads formed in the switch body 304. The open-ended channel 314 defines an inner surface 316, not shown in this view, and an outer surface 318. The switch body 304 is primarily composed of dielectric or nonconductive materials, such as a polymer or ceramic material. However, the outer surface 318 of the switch body 304 includes an outer conductive band 320 and an outer conductive surface 322, each engaged to the outer surface 318 and extending around the circumference thereof. The outer conductive band 320 is electrically separated from the outer conductive surface 322 by a space 324, the space 324 being a portion of the outer surface 318 of the switch body 304 having no conductive material applied thereto.

The switch body 304 includes a working end 326 and a switch end 328. The outer surface 318 of the switch body 304 at the working end 326 includes an annular bulge or shelf 330. The annular shelf 330 defines an outer diameter of the working end 326 that is greater than the outer diameter at the switch end 328. The difference in the outer diameter at the annular shelf 330 and the outer diameter at the switch end 328 creates and maintains a gap 333 between the outer conductive shell 306 and the outer surface 318 of the switch body 304. More specifically, the difference in outer diameters creates a gap 333 between the inner surface of the outer conductive shell 306 and each of the outer conductive band 320 and the outer conductive surface 322 sufficient to maintain electrical separation therebetween.

The outer conductive shell 306 is preferably made from a lightweight conductive material that is resiliently deformable so that a user may close the gap 333 using a manual grasping force applied to the outer conductive shell 306, as will be detailed further below. In preferred embodiments, the outer conductive shell 306 is formed of a thin metal sheet material, such as aluminum or steel. In more elaborate embodiments, the outer conductive shell 306 may be enclosed or otherwise covered on its outer surface by a polymer or rubber material to provide the user with more grip. When in used with tattoo machines, the outer conductive shell 306 may be disposable as medical waste, ensuring the power switch 300 does not become cross-contaminated between tattooing sessions.

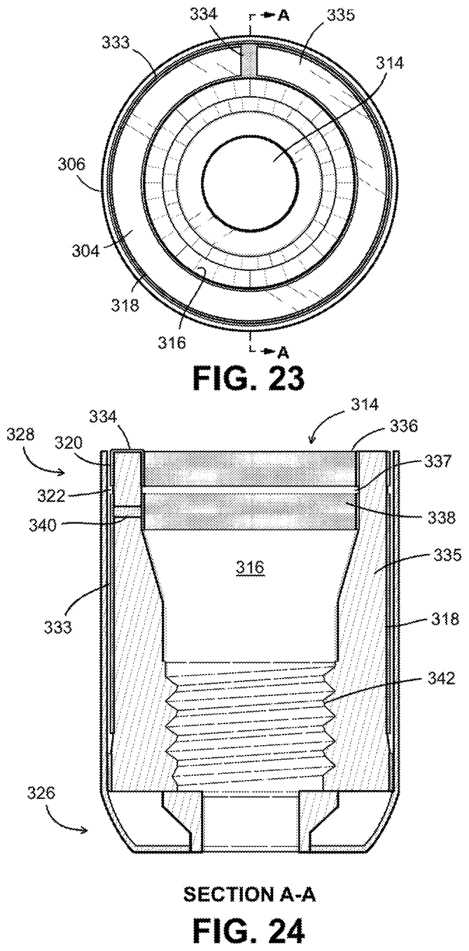

FIG. 23 is a top-down view of the switch body 304. More specifically, FIG. 23 is a top-down view looking into the open-ended channel 314 from the switch end 328 toward the working end 326. The gap 333 between the outer conductive shell 306 and the outer surface 318 of the switch body 304 is visible in this view. The gap 333 maintains electrical separation between the outer conductive shell 306 and the switch body 304. In some embodiments, a conductive strip 334 extends from the inner surface 316 to the outer surface 318 over a sidewall 335 of the open-ended channel 314. Note, the sidewall 335 of the channel 314 is shown in FIG. 23 as the thickness of the switch body 304. The sidewall 335 is separated from the outer conductive shell 306 by the gap 333.

FIG. 24 is a cross-sectional side view, taken along lines A-A marked in FIG. 23, of an embodiment of the switch body enclosed by the outer conductive shell. The inner surface 316 of the switch body 304 includes a lower conductive band 338 and an upper conductive band 336, each formed around the circumference of the inner surface 316 proximate the switch end 328. The lower conductive band 338 is electrically separated from the upper conductive band 336 by space 337, which is merely a part of the inner surface 316 having no conductive material applied thereto.

In preferred embodiments, the conductive strip 334 electrically couples the upper conductive band 336 to the outer conductive band 320. In similar fashion, a conductive pin 340 extends through the sidewall 335 of the channel 314 to electrically couple the lower conductive band 338 to the outer conductive surface 322. The two pins 332 extending from the tool body 302 contact the lower conductive band 338 and the upper conductive band 336. More specifically, one of the two pins 332 contacts the lower conductive band 338 and the other of the two pins 332 contacts the upper conductive band 336.

In alternative embodiments, the conductive strip 334 may be a second conductive pin extending through the sidewall 335, as opposed to over it. The second conductive pin would similarly electrically couple the upper conductive band 336 with the outer conductive band 320.

The working end 326 of the switch body 304 further includes internal threads 342 complimentary to the external threads 312 formed about the distal end 310 of the tool body 302. The internal threads 342 define the length L of the working end 326. In preferred embodiments, the two pins 332 are configured to respectively contact one of the lower conductive band 338 and the upper conductive band 336 when the tool body 302 is engaged along any length L of the working end 326 of the switch body 304. The length L of the working end 326 allows a user to easily adjust the needle length while maintaining the necessary electrical connections to power the motor on and off, similar to that described above with regard to FIGS. 19A and 19B.

Operation of the power switch 300 is similar to the operation described elsewhere herein regarding other embodiments of the grasp-activated power switch according to the present invention. The distal end 310 of the tool body 302 is threadably engaged to the internal threads 342 in the open-ended channel 314 at the working end 326 of the switch body 304. The outer conductive shell 306 substantially encloses the switch body 304. When assembled as such, one of the two pins 332 contacts the lower conductive band 338 and the other of the two pins 332 contacts the upper conductive band 336. To power the motor 307 off and on, a manual grasping force is applied to the outer conductive shell 306 at the switch end 328 of the switch body 304. The grasping force temporarily deforms the outer conductive shell 306 to close the gap 333 thereby electrically bridging the outer conductive surface 322 with the outer conductive band 320, which in turn generates a change of state pulse to power the motor 307 off and on.

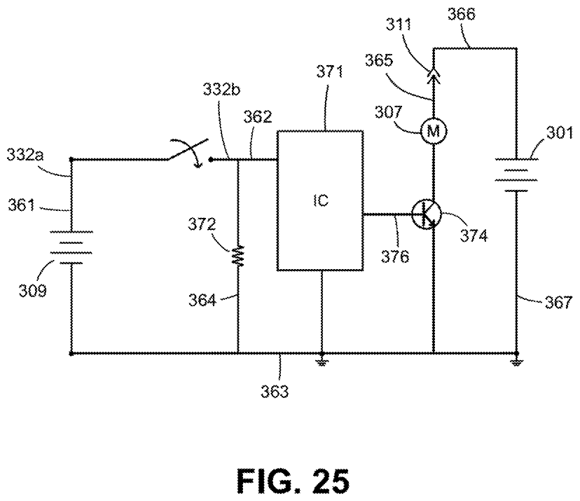

FIG. 25 is a circuit diagram illustrating the electrical connections according to one embodiment of the power switch 300. Wiring 361 connects a positive terminal of the battery 309 to one of the two pins 332, e.g., the first pin 332a. The other of the two pins 332, e.g., the second pin 332b, is connected by wiring 362 to the motor 307 and the negative terminal of the battery 309. More specifically, wiring 362 connects the second pin 332b with a positive terminal of an integrated circuit (IC) 371. Integrated circuit 371 is similar to the integrated circuits 71 and 271 previously described, i.e., configured as a toggle switch flip-flop. Wiring 363 grounds the negative terminal of the integrated circuit 371 with the negative terminal of the battery 309. In some preferred embodiments, an LED indicator 372 is connected in parallel by wiring 364 with the integrated circuit 371. When the power switch 300 is grasped by a user, the outer conductive shell 306 contacts the outer conductive surface 322 and the outer conductive band 320 to close the circuit between the two pins 332a and 332b generating a pulse that is fed as an input signal into the integrated circuit 371 which toggles the output between, for example, 0V and +5V. The toggling output pin is coupled to the base 376 of a transistor 374 configured to behave as a sold state switch. The transistor 374 changes state with each successive pulse, alternately enabling and disabling current flow to a relay coupled to a normally open switch. Thus, when current flow through the transistor 374, the relay is energized thereby closing the switch to energize the motor 309.

In some preferred embodiments, the motor is further connected to the power source coupling means 311 by wiring 365. The external battery pack 301 is similarly connected to the power source coupling means 311. Wiring 366 connects the positive terminal of the battery pack 301 to one side of the power source coupling means 311 and wiring 367 grounds the negative terminal of the battery pack. When the user completes the electric circuit, the change of state pulse generated turns the battery pack 301 off and on, which when turned on provides the necessary power to the motor 307 to sustain continued operation of the motor 307.

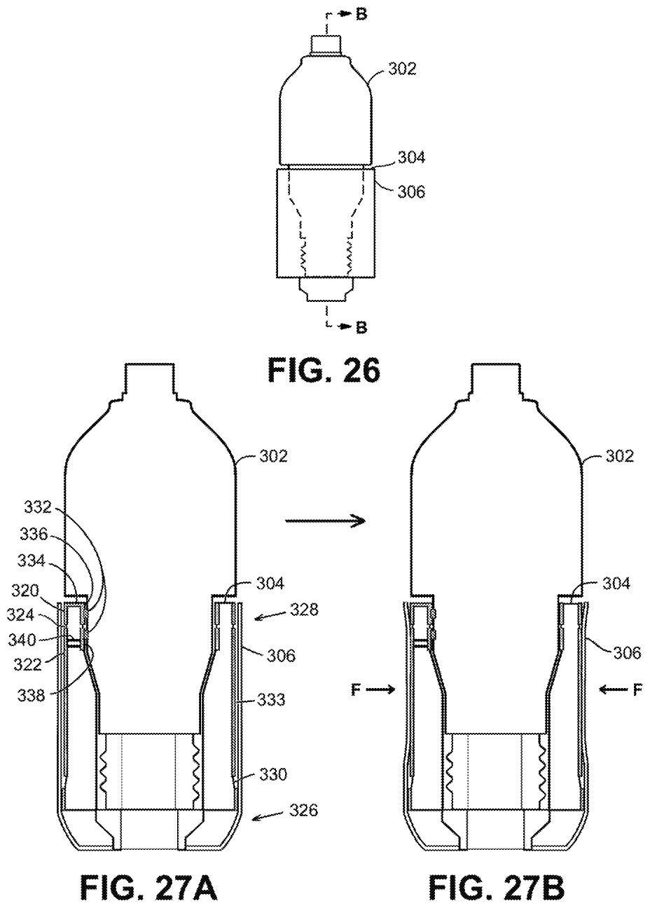

FIG. 26 is a partially transparent side view of an embodiment of the power switch assembled together. FIGS. 27A and 27B are side cross-sectional views, taken along lines B-B marked in FIG. 26, demonstrating a closing of the gap when a grasping force is applied to the outer conductive shell. The gap 333 is a result of the interaction of the outer conductive shell 306 with the annular shelf 330 formed around the working end 326 of the switch body 304. The gap 333 creates and maintains the electrical separation between the outer conductive shell 306 and each of the outer conductive band 320 and the outer conductive surface 322, as shown in FIG. 27A. When a user applies a grasping force F to the outer conductive shell 306, the shell temporarily deforms to close the gap 333 and thereby completing the electrical connection between the outer conductive surface 322 and the outer conductive band 320, as shown in FIG. 27B. Upon completing the electrical connection between the outer conductive surface 322 and the outer conductive band 320, a change of state pulse is generated to power the motor off and on, as described above.

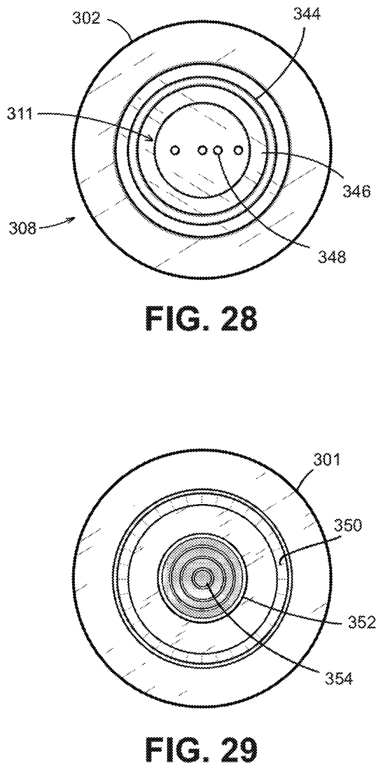

FIG. 28 is a proximal end view of an embodiment of the tool body according to the present invention. The proximal end 308 of the tool body 302 includes the power source coupling means 311. In preferred embodiments, the power source coupling means 311 includes a protrusion 344 extending from the proximal end 308. The protrusion 344 seats a magnetic ring 346 and a four pin connector 348. The motor 307 is electrically coupled to the power source coupling means 311 by internal wiring. FIG. 29 is a distal end view of an embodiment of a battery pack configured to engage the power source coupling means shown in FIG. 28. The distal end of the battery pack 301 thus includes an aperture 350 configured to receive the protrusion 344. The depth of the receiving aperture 350 is substantially equal to the length or height of the protrusion 344 off the distal end 308 of the tool body 302. Positioned at the center of the receiving aperture 350 is a second magnetic ring 352 enclosing a conductor 354. The battery pack 301 is coupled to the conductor 354 by internal wiring, as discussed briefly above. When assembled, the four pin connector 348 engages the conductor 354 to electrically couple the battery pack 301 to the tool body 302. The magnetic rings 346 and 352 ensure the electrical connection between the four pin connector 348 and the conductor 354 is maintained and allows a user to rotate the battery pack 301 relative to power switch 300 without disrupting the electrical connection therebetween.

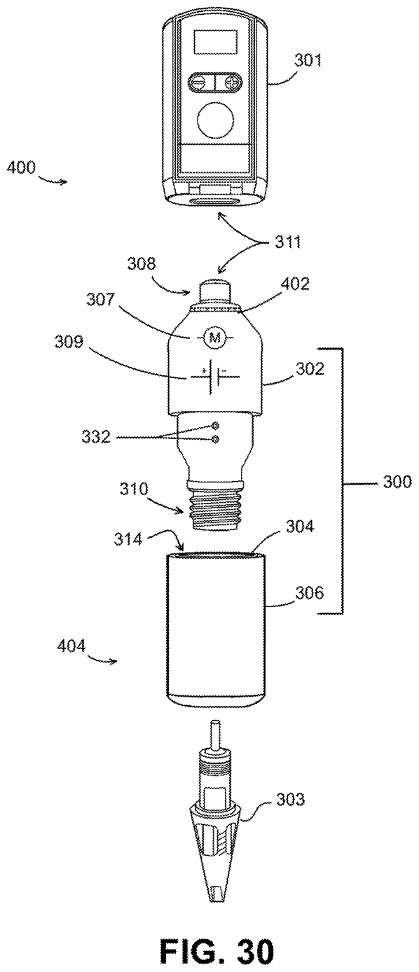

In more elaborate embodiments, the present invention may include a visual indicator, such as an illuminating filament or light-emitted diode (“LED”) band integrated into the tool body or the removable battery pack. FIG. 30 is an exploded side view of one such embodiment in the form of a power tool 400, which in this example is a tattoo machine. In alternative embodiments, the power tool 400 may be embodied as various other handheld power tools, such as hair clippers, trimmers, surgical instruments, or dental instruments. Power tool 400 has many of the same features as the handheld power tools described elsewhere in this application. In preferred embodiments, the power tool 400 utilizes the grasp-activated power switch 300 described above with regard to FIGS. 21-29. It should be understood that the visual indicator may be incorporated into power tools utilizing other embodiments of the grasp-activated power switch disclosed herein, e.g., the power switch shown and described with regard to FIGS. 1-20.

The primary difference between power tool 400 and the grasp-activated power switch 300 is the integration of the visual indicator or illuminating filament 402 into the tool body 302. Preferably, the illuminating filament 402 is integrated at the proximal end 308 of the tool body 302, opposite the grip 404 of the power tool 400, the grip 404 being made up of the switch body 304 and the outer conductive shell 306 that attaches to the distal end 310 of the tool body 302.

The illuminating filament 402 may be made of a plurality of light-emitting diodes (“LED”), or other known powered light sources. The illuminating filament 402 may partially or completely circumvolve the tool body 302 or the battery pack 301, depending on the embodiment. Importantly, the illuminating filament 402 is positioned opposite the working end of the tool body 302 so as to not be obstructed by an operator's hand during use. The position of illuminating filament 402 is selected so as to be continuously visible regardless of an operator's handling of the tool.

As will be discussed further below, the illuminating filament 402 is powered by battery 309, which is integral with the tool body 302, similar to motor 307. When the electric circuit is completed by applying a manual grasping force to the outer conductive shell 306, switch 300 closes, and the illuminating filament 402 changes state as described above with regard to the motor 307. The illuminating filament 402 and the motor 307 are electrically connected in parallel so that when the motor 307 is powered on, filament 402 illuminates; and when the motor 307 is powered off, filament 402 extinguishes.

Filament 402 may operate at two different lumen outputs, e.g., a first lumen output and a second lumen output. The first lumen output may be greater than the second lumen output. In preferred embodiments, upon initially changing state, i.e., powering on or powering off, filament 402 illuminates at a high lumen output for a preset short period of time, e.g., one second, coincident with manual actuation of switch 300. When going from a powered off state to a powered on state, and after the period of high lumen output, filament 402 dims by reverting to a low lumen output, i.e., the second lumen output, for as long as motor 307 runs continuously in the powered on state. The momentary high lumen output of filament 402 whenever switch 300 is actuated provides the operator with visual indicator of the changing state of the power tool 400. This is particularly beneficial when the power tool 400 is embodied as a tattoo machine where the machine is frequently powered on and off during a single tattooing session. The high lumen output coincident with filament 402 changing state is also beneficial in scenarios such as a surgical operating room or a dental office where the operator must know the state of the power tool 400 at all times when performing delicate operations. The high lumen output upon changing state provides an easy visual cue that the power tool 400 has changed state, i.e., been powered on or powered off.

Preferably, the first and the second lumen level of filament 402 each produces a light that is visible through several barriers of partially transparent protective material that may be applied to the tool body 302 and the battery pack 301. For example, when the power tool 400 is embodied as a tattoo machine, it is common for the tattoo artist to wrap at least the tool body 302 with a medical grade tape, such as cohesive bandage wrap, or other types of sterile barriers, such as a plastic sleeve, to prevent the tool body 302 from becoming contaminated during a tattooing session. The lumen output of filament 402, even at the second or lower lumen level is still bright enough to penetrate at least several layers of this sterile barrier. Common types of cohesive bandage wrap used in tattooing environments are opaque yet porous. Filament 402 preferably produces a lumen output that can penetrates two or more layers of the cohesive bandage wrap so that the light output is visible to the naked eye.

When present, the battery pack 301 may provide continuous power to both the motor 307 and to filament 402. In one variation, filament 402 is powered by battery 309 that is integrated within the tool body 302; however, adding load to battery 309 would require a battery with higher capacity, and consequently larger size, which conflicts with a design preference to minimize battery size at that location. Therefore it is preferable to utilize the higher capacity battery pack 301 as the source for both motor 307 and filament 402.

Filament 402 may partially or entirely circumvolve the tool body 302. In preferred embodiments, filament 402 fully circumvolves the tool body 302 to ensure the operator has visibility of the filament regardless of how the power tool 400 is positioned during use. Preferably, filament 402 is embedded in the outer surface and hermetically sealed to the tool body 302 ensuring contaminants cannot enter.

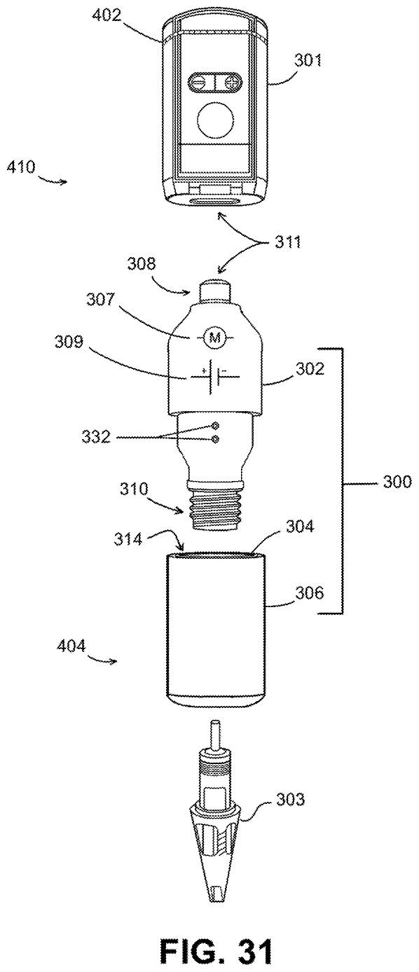

FIG. 31 is an exploded side view of an alternative embodiment of a power tool 410 according to the present invention. Power tool 410 illustrated by FIG. 31 is substantially similar to the power tool 400 discussed above. The difference is that illuminating filament 402 has been integrated into the battery pack 301 rather than the tool body 302. Filament 402 integrated into the outer surface of the battery pack 301 provides further separation from the grip 404 of the grasp-activated switch 300. Similar to power tool 400 discussed above, filament 402 is hermetically sealed within the outer surface of the battery pack 301. Filament 402 may also partially or fully circumvolve the outer surface of the battery pack 301.

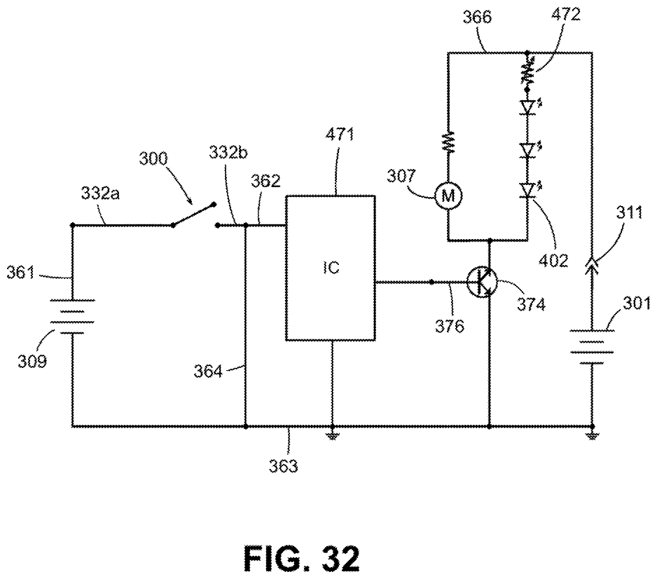

FIG. 32 is a circuit diagram illustrating one example of a control scheme that can be utilized in power tool 400 or 410. FIG. 32 is substantially similar to FIG. 25 discussed above and thus the basics will not be repeated in detail here. The primary difference between FIG. 32 and FIG. 25 is found in the addition of filament 402 to the circuit.

Further, the integrated circuit 371 has been replaced with integrated circuit (IC) 471. IC 471 may include a controller (not shown). The controller of IC 471 is configured to vary current flow through filament 402, e.g., by a variable current source or variable resistance 472 (modeled as a separate component external to IC 471 for convenience), which can be used to produce the different lumen output discussed above. In one embodiment, the variable resistance 472 may be a digital potentiometer configured within IC 471.

Briefly stated, when the grasping force is applied to the grip 404 and the electrical connection between 332a and 332b is completed, switch 300 closes and input 362 of IC 471 is energized, which raises or lowers the voltage of the base 376 of transistor 374, turning the transistor either on or off. IC 471 is configured such that a momentary pulse of voltage at input 362, caused by either momentary or prolonged closure of the grasp activated switch 300, will cause a change of state in voltage applied to base 376. Transistor 374 will remain in that state until switch 300 is opened and again closed. In this way, transistor 374 changes state with each successive cycling of switch 300, thereby alternately energizing and deenergizing base 376. Each time base 376 is energized, transistor 374 turns on to complete the circuit on the right side of the figure, allowing battery 301 to energize motor 307 and filament 402. Each time base 376 is deenergized, transistor turns off to open the circuit and shut off power to motor 307 and filament 402.

In preferred embodiments, the illuminating filament 402 may be a light-emitting diode (“LED”) band 402. The LED band 402 may be an addressable LED band, having integrated microcontrollers, or a non-addressable LED band, both of which are well known. The integrated circuit 471 may be configured to send a first pulse causing the LED band 402 to emit at the high lumen output upon the manual closure of switch 300. Shortly after the power tool 400 or 410 is powered on, e.g., after about one second, the LED band 402 may revert to the low lumen output after the initial high lumen display.

In some embodiments, a main controller is included as part of the integrated circuit 471. The main controller via the integrated circuit 471 may include logic for changing the color of the LED band 402 based on the duration the manual grasping force applied to the power switch 300. In some embodiments, the main controller via the integrated circuit 471 has logic to differentiate between a first grasping force and a second grasping force applied to the power switch 300, the first and second grasping forces being differentiated based on the duration of time an operator applies the manual grasping force to the power switch 300.

For example, a main controller may have logic to differentiate between a short duration manual grasping force, i.e., a first manual grasping force, and a prolonged duration grasping force, i.e., a second manual grasping force. The short duration grasping force may correspond power on and power off signals so that a quick grasping force, e.g., one second, applied to the power switch 300 changes the state of the motor 307 and the LED band 402, i.e., turns the motor and LED band on or off. With the motor 307 and LED band 402 powered on, a prolonged or continuous grasping force, e.g., three seconds or more, applied to the power switch 300 may be recognized by the main controller and cause the controller to command the LED band 402 to change color. There may be a series of preset colors stored within the memory of main controller and which are communicated to the LED band 402. Based on the signals received from the main controller via the integrated circuit 471 during application of the second manual grasping force, the LED band 402 may begin changing color until the grasping force is released from the power switch 300, e.g., once the operator has arrived at the desired color and released the power switch 300.

In embodiments utilizing an addressable LED band 402, the microcontroller integrated into the LED band 402 may store the last set color so that upon powering off and back on, the LED band 402 begins with the last set color. Similarly, the addressable LED band 402 may indicate the status of the energy stored by the battery pack 301 and change colors in response to the energy level according to known techniques.

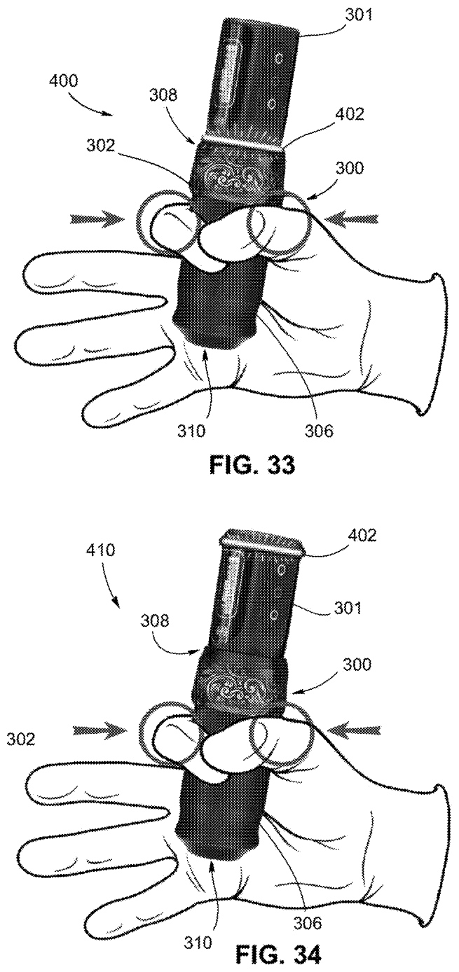

FIG. 33 is a perspective view of a power tool 400 changing state and FIG. 34 is another perspective view of a power tool 410 changing state. In FIGS. 33 and 34, the operator is applying the manual grasping force to the grasp-activated power switch 300 to either power on or power off the power tool 400 (FIG. 33) or power tool 410 (FIG. 34). As mentioned above, when the grasping force is initially applied to the switch 300 and the power tool 400 or 410 changes state, the filament 402 illuminates briefly at a first lumen output, e.g., one second at a high lumen output. This provides the operator with a visual indicator via the filament 402 that the power tool 400 or 410 has changed state, i.e., powered on or powered off.

Additionally, as can be seen in FIGS. 33-34, the filament 402 is positioned remote from the switch 300 so that the filament 402 is unobstructed by the operator's hand when using the power tool 400 or 410. In power tool 400, this means the filament 402 is placed at near the proximal end 308 of the tool body 302. In power tool 410, the filament 402 can be placed anywhere along the battery pack 301. In some preferred embodiments of power tool 410, the filament 402 is placed at the most remote location from the tool body 302, e.g., around the upper end as shown.

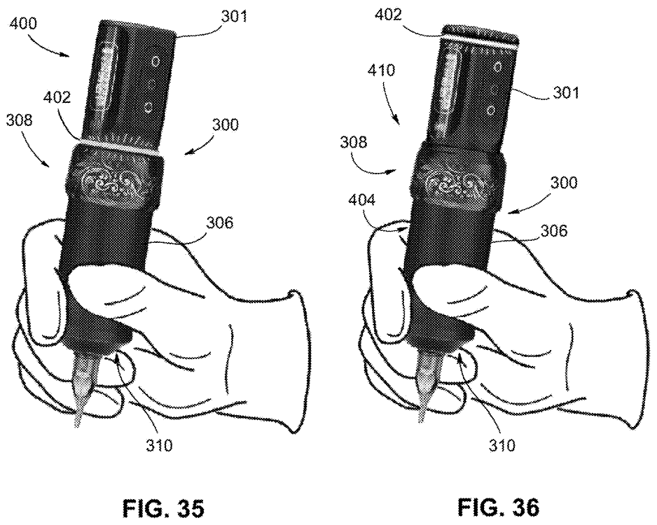

FIG. 35 is a perspective view of a power tool 400 in use and FIG. 36 is a perspective view of power tool 410 in use. In contrast to FIGS. 33-34 discussed above, FIGS. 35-36 illustrate the power tool 400 and 410 after the grasping force has been applied to the switch 300 and the tool 400 and 410 is in a powered on state. The filament 402 has reverted to the second lumen output, i.e., low lumen output, to indicate to the operator that the power tool 400 and 410 is in a powered on state. The operator may hold the power tool 400 and 410 by grasping the grip 404 portion of the switch 300, which leaves the filament 402 visible during use.

Exemplary embodiments of the invention have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that that scope shall not be restricted, except in light of the appended claims and their equivalents.

What is claimed is:

1. A handheld power tool, comprising:
a tool body enclosing a motor;
a grip removably attachable to the tool body, the grip having a grasp-activated switch;
a battery removably attachable to the tool body; and
an illuminating filament at least partially circumvolving one of the tool body or the battery;
wherein, in response to a manual grasping force applied to the grip, the grasp-activated switch electrically couples the battery to the motor and to the illuminating filament.

2. The power tool of claim 1, wherein the battery, upon the manual grasping force being applied to the grasp-activated switch, generates a pulse causing the motor and the illuminating filament to change state.

3. The power tool of claim 1, wherein the battery, in response to the manual grasping force, is configured to maintain the motor and the illuminating filament in an on state or an off state.

4. The power tool of claim 1, wherein the illuminating filament has a first lumens output and a second lumens output.

5. The power tool of claim 4, wherein the first lumens output is greater than the second lumens output.

6. The power tool of claim 5, wherein, when the battery is electrically coupled to the illuminating filament in response to the manual grasping force, the illuminating filament produces the first lumens output for a preset period of time upon initially changing state.

7. The power tool of claim 5, wherein the illuminating filament produces the second lumens output when maintained in a powered on state.

8. The power tool of claim 1, wherein the illuminating filament fully circumvolves the battery.

9. The power tool of claim 1, wherein the illuminating filament fully circumvolves the tool body at an end opposite the grip.

10. The power tool of claim 1, wherein the illuminating filament comprises a light-emitting diode band.

11. The power tool of claim 10, wherein the light-emitting diode band is configured to change color in response to a stored energy level of the battery.

12. The power tool of claim 10, further comprising a controller electrically coupled to the battery and to the illuminating filament, the controller configured to differentiate, based on a duration the manual grasping force is applied, between a first manual grasping force and a second manual grasping force, and command the light-emitting diode band to change color in response to the second manual grasping force.

13. The power tool of claim 12, wherein, when the second manual grasping force is applied, the controller commands the light-emitting diode band to change color among a series of preset color options.

14. The power tool of claim 1, wherein the illuminating filament is electrically isolated from the motor.

15. The power tool of claim 1, wherein the illuminating filament is embedded in the tool body.

16. The power tool of claim 1, wherein the illuminating filament is hermetically sealed.

17. The power tool of claim 1, wherein the power tool is selected from a group of power tools consisting of: a tattoo machine, a hair clipper, a surgical instrument, or a dental instrument.

18. A handheld power tool, comprising:
a tool body enclosing a motor, the tool body having a distal end, a proximal end, a first pin and a second pin, the first pin and the second pin extending from an outer surface of the tool body;
a grasp-activated power switch having an inner body removably attachable to the distal end of the tool body, and an outer conductive shell substantially covering the inner body, the inner body having a working end, a switch end, and an open-ended channel extending between the working end and the switch end, the channel configured to receive the distal end of the tool body and defining an inner surface electrically coupled to an outer surface of the switch body, wherein the inner surface is electrically separated from the first pin and the second pin;
a battery removably attachable to the proximal end of the tool body; and
an illuminating filament at least partially circumvolving one of the tool body or the battery;
wherein a manual grasping force applied to the grasp-activated power switch completes an electrical connection between the first pin and the second pin thereby energizing the motor and powering the illuminating filament.

19. The power tool of claim 18, wherein the illuminating filament comprises a light-emitting diode band.

20. The power tool of claim 19, wherein the light-emitting diode band fully circumvolves one of the tool body or the battery.
